(19) United States
(12) Patent Application Publication
(10) Pub. No.: US 2020/0022637 A1
(43) Pub. Date: Jan. 23, 2020

(54) SYSTEM, DEVICE AND METHOD FOR BLADDER VOLUME SENSING

(71) Applicant: The Regents of the University of California, Oakland, CA (US)

(72) Inventors: Eric A. Kurzrock, Davis, CA (US); Soheil Ghiasi, Davis, CA (US)

(21) Appl. No.: 16/576,590

(22) Filed: Sep. 19, 2019

Related U.S. Application Data
(63) Continuation of application No. PCT/US2018/020972, filed on Mar. 5, 2018.
(60) Provisional application No. 62/476,654, filed on Mar. 24, 2017.

(51) Int. Cl. A61B 5/20; A61B 5/00; G01N 21/47

(52) U.S. Cl. CPC A61B 5/208; A61B 5/6833; A61B 5/0075; G01N 2201/062; A61B 5/7267; G01N 21/4738; A61B 5/6823

(57) ABSTRACT

Here discloses a system for sensing bladder volume. The system includes at least one patch, a plurality of light emitters, a plurality of light sensors, and a process. The at least one patch is configured to attach to a human skin or a wearable garment at locations in proximity to an abdomen area. The light emitters are directed towards the abdomen area. The light sensors are configured to receive light signals that are emitted by the light emitters, reflected by human tissues, and transmitted through an abdominal wall. At least one of the light emitters or at least one of the light sensors is disposed on the at least one patch. The processor is configured to receive information of the received light signals and to predict a bladder volume based on the information of the received light signals.

### CROSS-REFERENCE TO RELATED APPLICATIONS

This application is a continuation of International Application No. PCT/US2018/020972, filed Mar. 5, 2018, which in turn claims priority under 35 U.S.C. § 119(e) to U.S. Provisional Application No. 62/476,654, filed Mar. 24, 2017, the contents of each of which are incorporated herein by reference in their entireties.

### BACKGROUND

Many patients suffer from the consequences of spinal cord injury (SCI) and congenital spinal anomalies such as spina bifida. In 2016 for example, about 300,000 SCI patients lived in the US alone. Although these patients tend to have obvious limitations in mobility, unbeknownst to the general public is that nearly all of them have neurogenic bladder dysfunction and lack sensation and control of their bladder. The majority of these individuals have to perform clean intermittent catheterization (CIC), which is either self- or assisted urethral catheterization for drainage of the bladder on a defined schedule to minimize the detrimental effects of elevated bladder pressure such as hydronephrosis, vesicoureteral reflux, infections, renal scarring, and ultimately, progression to end stage renal disease.

Although prevention of renal failure is of paramount importance, this is not the day-to-day concern of most patients. Their biggest concern is continence. Without timely CIC they can leak urine and most need to wear some form of protection, such as an adult diaper. Some patients go as far as to choose a permanent indwelling catheter which leads to chronic infection and a higher risk of bladder cancer. Incontinence can have major impacts on patients' social and mental health, and thus, on their care costs and quality of life.

Since SCI patients are unable to sense bladder fullness, current recommendations typically involve catheterization every 2 to 4 hours throughout the day. This high frequency of emptying adds “insult to injury.” A commonly reported problem is making the difficult trip to the bathroom and only finding a small amount of urine in the bladder, or worse, not getting to the bathroom in time and leaking because the bladder was too full. In reality, it is not time that dictates the need for bladder emptying; it is the bladder volume.

### SUMMARY

The technology disclosed herein replaces the current time-triggered catheterization regimen with volume-triggered (demand-based) timely catheterization. If SCI patients are given timely alerts, they can plan bathroom trips accordingly, decrease the frequency of emptying, improve compliance, protect their kidneys, avoid incontinence, increase social activities, and ultimately, improve their quality of life.

At least some embodiments of the disclosed technology relate to a system for sensing bladder volume. The system includes at least one patch, a plurality of light emitters, a plurality of light sensors, and a processor. The at least one patch is configured to attach to a human skin or a wearable garment at locations in proximity to an abdomen area. The light emitters are directed towards the abdomen area. The light sensors are configured to receive light signals that are emitted by the light emitters, reflected by human tissues, and transmitted through an abdominal wall. At least one of the light emitters or at least one of the light sensors is disposed on the at least one patch. The processor is configured to receive information of the received light signals and to predict a bladder volume based on the information of the received light signals.

### BRIEF DESCRIPTION OF THE DRAWINGS

FIG. 1 illustrates typical urodynamic of bladder pressure (in cm of water) vs volume of injected water (in ml) for normal and SCI subjects.

FIG. 2 illustrates mechanisms using near-infrared spectroscopy (NIRS) for a) probe swept across the pelvic area, b) characteristic profile of the sensed intensity, c) scheme of a probe with multiple light source and detectors.

FIG. 3 illustrates a sample device with a probe with one LED-photodetector.

FIG. 4A illustrates a bladder phantom with a balloon.

FIG. 4B illustrates sensed diffuse light as the probe is swept across the phantom, where line corresponds to a different bladder volume.

FIG. 5 illustrates a bladder phantom with meat.

FIG. 6 illustrates diffuse intensity as a function of probe location relative to a larger container.

FIG. 7 illustrates a wearable sensing device and its application on a human subject, where a computer is interfaced with the wearable sensing device to capture and virtualize the data.

FIG. 8 illustrates diffuse intensity (volts) sensed at multiple photodetectors when the corresponding light emitting diode is turned on, before and after voiding the bladder.

FIG. 9 illustrates a sensing system that includes a non-invasive optical probe patch, electronic circuitry, and a software module for analyzing collected data.

FIG. 10 illustrates a comparison between radiance and irradiance limits and the sensing system.

FIG. 11 illustrates a sample scenario that the sensing system is used to monitor a patient's bladder.

FIG. 12 illustrates a sample architecture of a bladder volume sensing system.

FIG. 13 illustrates a high-level overview of a control logic.

FIG. 14 illustrates an LED Driver IC and a Digital Rheostat IC that can be used together to achieve dynamic LED sink current levels.

FIG. 15 illustrates an LED selective network.

FIG. 16 illustrates a sample integrated circuit (IC) that controls photodiodes.

FIG. 17 schematically illustrates an experimental setup including an optical phantom.

FIG. 18 illustrates an ex vivo experimental setup using a pig bladder and intestines to create a tissue model.

FIG. 19 illustrates measurements performed on the optical phantom over three wavelengths.

FIG. 20 illustrates measurements performed on the optical phantom over three volumes of liquid.

FIG. 21 illustrates the ex vivo measurements performed on a tissue model including a pig bladder.

FIG. 22 is a high-level block diagram illustrating an example of a hardware architecture of a computing device that may perform various processes as disclosed.

### DETAILED DESCRIPTION

The disclosed technology relates to a non-invasive, small cyber-physical system (CPS) device whose patch-like sensing probe is worn on the lower abdomen in the pelvic area. The probe has one or more near infrared (NIR) light emitting diodes (LEDs) and photodetectors. The probe is controlled by a tiny embedded computer, with computing, storage, analog interfacing and wireless communication capabilities, which can sit on top of the sensing probe. The embedded system can run on a small battery, and can execute sophisticated data analysis algorithms to accurately estimate bladder volume. The disclosed technology leverages the principles of near infrared spectroscopy (NIRS) to directly correlate diffuse optical signal at, e.g., about 975 nm (for which water is highly absorptive) to bladder volume, bypassing the need to solve an inverse imaging problem.

The analytics engine of the disclosed technology can proactively trigger measurements, can incrementally improve as the patients use the device, and can automatically personalize the prediction algorithm to better serve the patient's specific situation. The entire system can be easily hidden under patient's normal clothing. The disclosed system can estimate the bladder volume with target accuracy of 25 ml, and alerts SCI patients in time to start looking for a bathroom to perform CIC.

The alert feedback mechanism to close the feedback loop can be implemented in a number of possible ways. Some examples include alerts on the device (e.g., a discreet ringtone or vibration) for device self-sufficiency and ease of use; alerts shown on the patient's smart devices (e.g., a smartwatch or a smartphone); alerts implemented by a dedicated unignorable alarm (e.g., a vibrating wristband that can be turned off only by the device as the bladder volume reduces: mimicking natural feeling of urge-to-urinate which is relieved only as the bladder is being emptied); or sent to patient's caregivers. The optimal strategy can depend on the patient preferences and his/her specific circumstances.

In some embodiments, the system includes one or more near infrared light emitter(s) that send pulses of light through the abdominal wall and one or more sensor(s) detect the reflection of light. An array of lights can be placed on the body with an adhesive or incorporated into a wearable garment such as a belt or girdle. Similarly, the sensors can be incorporated into the same garment, another garment or adhesive patch. The lights can be activated individually or in combinations. Likewise, one or more sensors can be activated individually or in combinations.

In some embodiments, the system including the light emitters and sensors can be implemented as a wearable device that can be physically attached to a patient body, such as a strap, a belt, a watch, a necklace, a wearable garment, etc. The device may include components that are cost-effective and can be implemented as a disposable device.

These devices can transmit information to a central processing unit (CPU) and receiver which can be outside the body or to an implantable device (placed under the skin). The receiver can itself process the information and determine the bladder volume or it can send the information wirelessly to another CPU such as a smart phone for processing in the phone or sent via cell signal or Wi-Fi for, e.g., cloud computing.

In some embodiments, the signals collected by the sensors (also referred to as telemetry signals) can be transmitted to a remote computing device (e.g., a smart wearable device, a smart phone, a tablet computer, a laptop computer, a cloud server, etc.) for further data processing. For example, the device attached to the patient body can include the light emitters, sensors and a data communication interface. The signal are transmitted via the data communication interface to the remote computing device for further analysis. In some embodiments, the data communication interface can be either wireless or wired. For example, the data communication interface can be a Bluetooth or Wi-Fi interface, or a wired Ethernet interface.

The pattern of light detected by the sensor(s) can be analyzed by software and compared to catheterized or voided volumes during a training period. Machine learning can be utilized to “customize” the light patterns for each patient's body and bladder. The software and hardware can be optimized to learn when to emit light to reduce power need. Variables that can be processed include environmental variables (e.g., ambient temperature, outside humidity) and information related to the patient (e.g., leak point volume, patient activity (entered by patient or motion sensor data from smart phone) and liquid intake). Either the receiver or smart phone can then notify the patient when a threshold volume is reached and signal that it is time to empty their bladder. This signal can be, e.g., auditory, visual or vibratory.

In some embodiments, all sensors in the array can record the reflection of light of each individual emitter. Patients may have their bladders filled via a catheter in small increments. After each increment, each light can be fired. All sensors can be turned on. Then, through machine learning, the system can optimize the pattern or customize for each patient and determine which sensors are the most predictive for each emitter in determining the volume. Various variables can also be used in the machine learning, such as environmental variables (e.g., ambient temperature and humidity gathered by GPS on smartphone) and patient activity information (e.g., oral liquid intake recorded by the patient into smartphone, and activity level determined by motion sensor on smartphone (similar to watches that measure steps)). All of these variables can be used to predict of urine production.

Bladder as a Physical System

A. Brief Overview of Anatomy and Physiology

The kidneys filter blood and remove electrolytes, some chemicals and excess water to produce urine. The urine is transported down the ureters by muscular contractions into the bladder. An empty bladder occupies a small volume, and is normally situated right behind the pubic bone in both male and female subjects. The bottom of bladder is relatively fixed in place, and is connected to the urethra.

As the bladder fills naturally with urine, its volume increases, and its dome and sides grow into the abdominal cavity. The bladder dome is fairly superficial and the tissue between the anterior wall of the bladder and abdominal skin, right above the pubic bone (where the device's probe can be placed), is about 2-5 cm thick for most patients. The bladder wall thickness is about an order of magnitude smaller in comparison (3.0±1 mm and 3.3±1 mm in normal adult female and male subjects, respectively).

The bladder normally stores urine at extremely low pressures (less than 5 cm of water). The muscles of the bladder accommodate the urine by passive relaxation controlled by the autonomic nervous system. In healthy subjects, even at close to full bladder capacity, the pressure remains low despite the strong urges to void. This low pressure allows the kidneys to continue their filtering function. On the contrary, many bladder diseases and neurologic disorders cause high storage pressures if the bladder volume increases past certain thresholds. High intravesical pressure is detrimental to kidneys, and can cause renal failure, and thus, it can be avoided.

When a neurologically intact patient decides to void, the brain (cerebrum) sends a signal to the pontine portion of the spinal cord that initiates a cascade of nervous signals and reflexes that allow volitional voiding. The bladder contracts and almost simultaneously the bladder neck and urethral sphincter open. This allows voiding to occur at pressures between 10 and 30 cm water (relatively low).

For most of persons, after the decision to void, the persons are not cognizant of the intricate processes and coordination that can occur. On rare occasion, the persons appreciate this miracle when someone walks into the bathroom and the persons purposefully close sphincter and hope the bladder stops contracting. Being “pee shy” is the converse situation. For the many patients with spinal cord injuries this less-than-appreciated process is very much a central point of their lives.

B. Relevant Dynamics of the Physical System

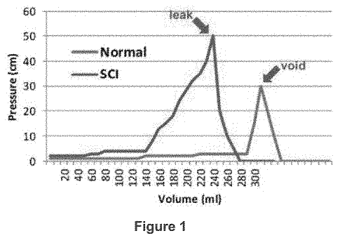

Urodynamic testing is a clinical procedure to measure bladder pressure at different fill volumes, and to quantitatively characterize the compliance and function of the bladder. During the test, the bladder is slowly filled with water via a dual-lumen urethral catheter while measuring the intravesical pressure. The pressure is then plotted against total volume of instilled water. FIG. 1 illustrates a typical bladder filling curve of a healthy subject and an SCI patient. The X and Y axes show the volume of instilled water and the intravesical pressure, respectively. As water is instilled at a set rate, e.g., 20 ml per minutes, one may view X axis as test progression time with instilled volume annotations.

The normal bladder (blue line) shows no significant elevation of pressure (natural bladder stretch to protect the kidneys) as additional water is instilled into the bladder. When the patient is asked to volitionally void at his/her capacity, 300 ml for this patient, there is a brisk rise in bladder pressure. The bladder neck and urethral sphincter open in harmony, and the urine flows out without significant resistance.

The typical patient with SCI (red line) has a lower functional volume with a brisk rise in pressure at a low volume (150 ml in this example) without any warning or sensation. As the bladder is filled, intravesical pressure increases until the patient experiences an involuntary contraction (at about 240 ml) and leakage of urine at high pressure due to poor coordination between the bladder and sphincter. The sustained high pressure after 150 ml can be harmful to the kidneys. This SCI patient's safe volume is less than 150 ml and he/she can be best served by performing clean intermittent catheterization at about 150 ml volume to prevent kidney damage and incontinence.

Patients routinely undergo urodynamic testing to characterize their bladder's dynamics, and to know their safe volume. However, they are unable to sense their bladder fullness, and cannot reliably follow a prescribed safe-volume emptying schedule.

C. Appropriate Measure for Demand Detection: Bladder Volume

A key physiological reality is lack of reliance on time as a metric for sensing the urgency to void. Unlike urodynamic testing, human hydration and urine production do not occur at a constant rate, so the bladder can fill to functional capacity in less than an hour or in many hours.

In healthy individuals, functional capacity is directly sensed by cells that respond to stretch. Bladder stretch is strongly correlated with its volume, and the urge to void is sensed at individual-specific volumes while there is no significant pressure built up in the bladder.

There is sometimes the misconception that intravesical pressure can be used as a metric for voiding alarm generation. In practice, pressure fails to be an effective measure for several reasons. First and foremost, intravesical pressure is detrimental to kidneys, and material pressure rise in the bladder can be avoided, rather than relied upon as an alarm-generation metric. Also, pressure increase occurs close to the leak point for the majority of patients, i.e., their urodynamic curve rises with a sharp slope. In those cases, pressure-triggered alerts cannot provide sufficient warning time for patients to make a trip to the bathroom and place a catheter. Furthermore, it is difficult to distinguish intravesical from intraabdominal pressure due to movement, body position, coughing, sneezing and such.

The disclosed technology relates to a non-invasive bladder volume estimation mechanism to generate timely CIC alert feedbacks. If SCI patients are provided awareness of the volume of urine in their bladder in time, they can plan bathroom trips accordingly, decrease the frequency of emptying, improve compliance, protect their kidneys, and avoid incontinence.

Related Information

Currently, bladder volume is measured in the clinic using Doppler-ultrasound tomography. In an exam, bladder dimensions are measured from which the volume is estimated, assuming an elliptical bladder shape. Commercial Off-The-Shelf (COTS) ultrasound tomography devices are bulky, expensive, and their application requires training and skill. Furthermore, the time, effort, and privacy needed for a typical SCI patient to repeatedly set up the device and perform the procedure can be more intrusive to patient's daily life than just performing CIC instead.

A number of investigators have attempted to develop an implantable biosensor to measure bladder pressure. However, this strategy has critical shortcomings, and monitoring of pressure is not a plausible mean to avoid incontinence for most patients with neurogenic bladder.

Other attempts at providing chronic bladder volume measurements include implanting a magnet to interact with an external electronic switch to signal conformational changes in bladder shape due to volume. This invasive approach is subject to errors due to the long-term degradation of magnetic strength, interference from the Earth's and peripheral magnetic fields. Furthermore, its adoption is hampered by the patient's inability to have an MRI exam after implantation.

Others have used MEMs (Microelectromechanical systems)-enabled, implantable strain-gauge sensors to look at bladder size and correlate that to a known volume. These approaches face non-trivial concerns surrounding invasiveness, biocompatibility, telemetry, and power. Moreover, the bladder tissue is likely to develop fibrosis where the implant is attached. Fibrosis can change the physical properties of the tissue at the sensor location (e.g., it may not stretch like before), which can render the approach ineffective in the long term. That is, the sensor may significantly influence the system that it is trying to sense.

One non-invasive approach uses electrical-impedance tomography to estimate the conductance distribution of the pelvic region using a belt with multiple electrical contacts. The approach has not been successful in practice, partly due to unreliability of skin contacts, very low resolution in bladder volume estimation, and high variability in patient tissue composition, which makes up its impedance.

Near-infrared spectroscopy (NIRS) based systems measure the changing concentration of oxy and deoxy hemoglobin of the bladder wall, in which the most notable changes occur during voiding. However, SCI patients showed inconsistent trends in tissue oxygen saturation, likely due to the neurogenic bladder. Furthermore, chromophore changes that occur during voiding are not helpful in warning the patient when bladder volume increases.

Researchers leveraged NIRS-based bladder monitoring via a single light source-detector pair to determine full versus void states of the bladder by measuring the attenuation in light due to water. However, due to various noise artifacts, such as geometrical variation due to breathing and exact probe position, a reliable determination of different bladder states using the reported NIRS setup proved infeasible.

Despite multiple attempts at solving this problem by various engineers, there exists no practical solution for patients. Prior approaches failed to fully take into account cyber-physical and clinical considerations such as variability among patients' body types, long-term reliability requirement and ease of use.

In addition to spinal cord injury patients who have lost all control of the bladder, there are many more patients who suffer from bladder dysfunction. Examples include men with prostatic disease, women who have stress incontinence, children with congenital spinal cord anomalies, and diabetics who have lost bladder sensation. The pathophysiology of each of these disorders is different but ultimately these patients suffer from incontinence and/or damage to their kidneys when the bladder fills beyond a certain volume. Many of these patients do not have the ability to sense when they have reached that important bladder volume either due to a primary bladder disorder or a neurologic deficit. The disclosed technology is useful to all such patients.

Sensor Array Design for Non-Invasive Bladder Fill Assessment

The disclosed technology utilizes principles of near-infrared spectroscopy to estimate the subject's bladder volume. In the 650 nm to 900 nm range of spectra, most tissue chromophores have relatively low absorption. This allows enough light to traverse fairly deeply, on the order of several centimeters, and back scatter to be detectable non-invasively using commodity components. However, as wavelength increases past 900 nm the absorption coefficient of water steadily rises and several pronounced peaks quickly appear. The peak at ˜975 nm coincides with low absorption of other chromophores in the tissue, and is well suited for the target approach. Note that urine can include 91%-96% water.

A. Approach

By measuring the intensity of back-scattered light (diffuse reflectance) at ˜975 nm across the pelvic region, with fixed-spacing between light emitters and detectors, the spatial information about the bladder can be inferred, which can be mapped to volume numbers via prediction algorithms, which are discussed in details in following sections.

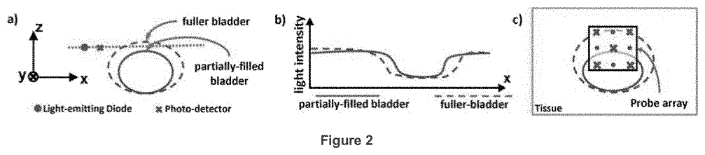

Assuming that there is a controllable NIR light field in the subject's abdominal cavity, right above the pubic bone, as the bladder expands, its growth into the light field causes additional light absorption, which can reduce the amount of back scattered light. FIG. 2 visualizes this idea. In FIG. 2.a a light source-detector probe, with fixed spacing between the source and detector, is assumed to sweep across the lower abdomen (on top of the bladder) on the dashed red line. FIG. 2.b shows the expected intensity of the diffused back scattered light at the detectors for the two different bladder volumes, as function of probe location on the red line. The idea is that larger bladder volumes can absorb more light.

A major challenge in leveraging this phenomenon to estimate bladder size is the substantial variability and indeterminism in the physical bladder system, which greatly impacts the light propagation. Patients have different tissue thickness and composition, body shape, bladder size and so on. Even dynamics such as breathing, diet and movements impact the physical system under consideration from a light propagation viewpoint. As a result, the actual measurements are noisy and unreliable.

An LED-detector probe with 3 cm fixed spacing can be placed on the lower abdomen of a healthy human subject during voiding. Light attenuation can be recorded as a function of time during which, the patient is asked to void, starts voiding, and ends voiding.

Two observations are made about the recorded data: 1) general decrease in light attenuation (higher sensed light intensity) as the bladder contracts, and 2) highly noisy nature of the signal. Additional experiments demonstrate that measurements with a single source-detector probe are too unreliable to yield an accurate alert generation criterion across voiding trials. The underlying issue is that if deployed, single-point measurements yield a noisy number (sensed light intensity) on every observation, which confuses threshold-based volume estimation algorithms.

According to the disclosed technology, noisy single probe measurements can be mapped to volume numbers with sufficient accuracy, if a large number of simultaneous measurements from across the pelvic area are available. FIG. 2.c sketches the idea, which is elaborated further in the following sections. FIG. 2.c shows a probe scheme that includes light sources and detectors with fixed spacing. The embedded system controlling the probe activates the light sources in sequence, and can collect measurements from all photodetectors for every light source. The rationale is that the reduction in diffuse reflectance gives rise to patterns across the detectors, and enables mapping bladder's spatial spread to its volume with sufficient accuracy

Correlated observations from the bladder through a multi-channel probe can yield distributed characteristic patterns, which unlike intensity values are robust to noise. This is critical since noise artifacts introduced by ambient light, breathing, motion, natural variations in body shape and probe placement are going to be norms, rather than exceptions. In addition, the disclosed technology can personalize the pattern recognition algorithms, via post-deployment fine-tuning of regression parameters based on user feedback, to better handle specific circumstances of each patient.

Unlike conventional imaging techniques such as coherent optical tomography, the disclosed technology does not need to solve an inverse problem, and directly estimates a single number (bladder volume) through predictive models. Therefore, the disclosed technology can manage to achieve the goal with dramatically simplified optical frontend and processing backend. Also the disclosed technology is different from conventional array sensing technique, e.g., radar phased arrays, array sensing assumes an isotropic field for wave propagation. Human body, however, is an anisotropic medium for photon migration.

B. Samples

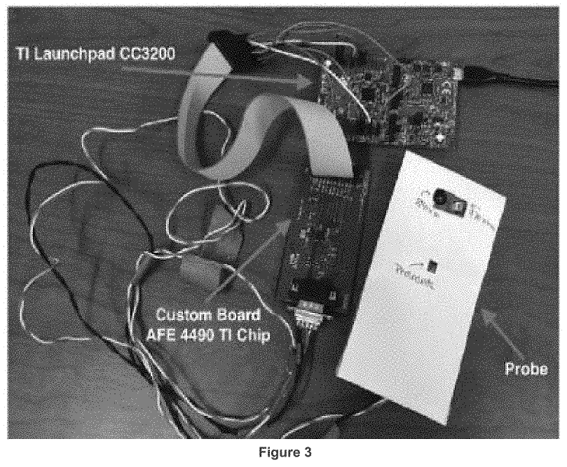

An example device is illustrated in FIG. 3. The device includes an optical probe (including one or more LEDs), interfacing electronics (e.g., custom board for TI AFE 4490 Chip), and an embedded computer (e.g., TI Launchpad CC3200) to control data acquisition. The device cycles LEDs on and off at high-frequency, and processes sensed intensity values to remove the effect of ambient light from measurements. The disclosed technology also includes a software interface for real-time recording, display and analysis of data. The optical probe uses a 970 nm Infrared LED and a photodiode to acquire diffuse optical signal. Two simple bladder phantoms are used to mimic the optical and some mechanical properties of a bladder.

First Sample

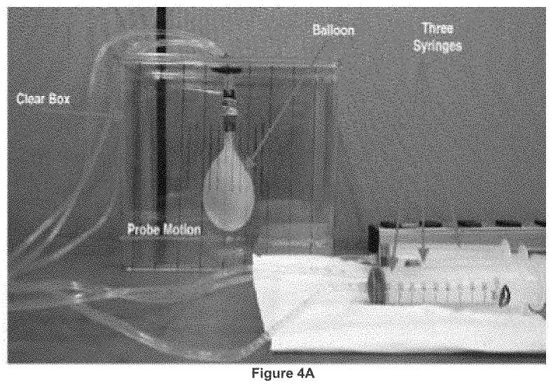

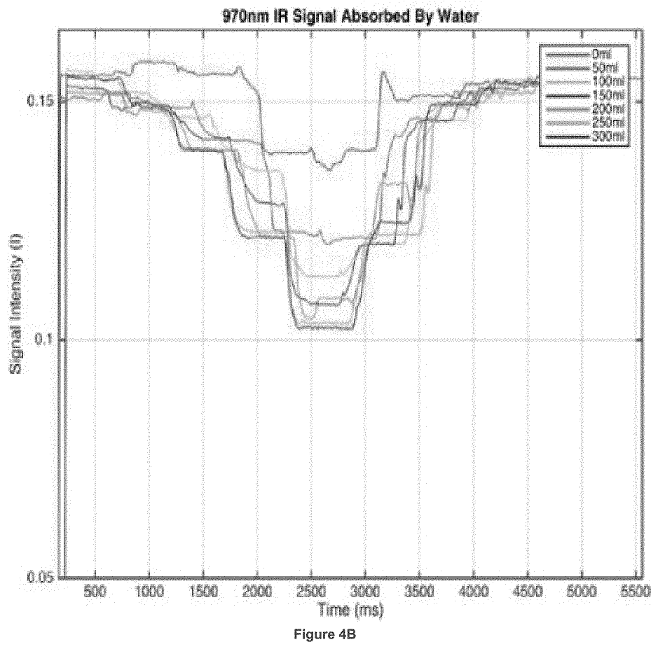

First bladder phantom includes a translucent latex balloon placed in the center of a clear box. A network of tubing and syringes enables inflating the balloon with water to emulate the bladder filling with urine (Urine is typically about 95% water). The phantom setup is shown in FIG. 4A.

The balloon is inflated with water, and then the prototype is used to record measurements of the diffused infrared light. Specifically, the balloon is filled at 50 ml increments up to 300 ml. At each increment, the optical probe is manually slid across the phantom at a steady slow pace, and voltage measurements (after trans-amplification by TI AFE 4490 and filtering out the impact of ambient light) are recorded. The voltage readings are sampled at 1 KHz, and each trial lasted about 5 second.

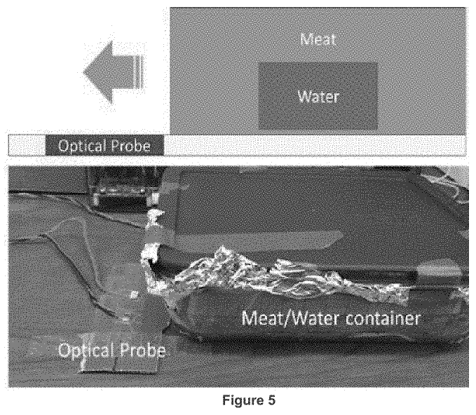

The raw data is visualized in FIG. 5. Due to manual sliding of the probe, sample time-stamps may be considered as merely correlated with rather than accurate predictor of probe location on the clear box. There are several observations. First, each experiment with a fixed volume balloon confirms increase in IR light attenuation (lower voltage) when the balloon sufficiently overlaps with the light field. Second, as the balloon increases in size, its overlap with the light field also expands: larger balloons occupy larger volumes and absorb more photons at a comparable location (e.g., when the source-detector pair is placed directly across from the balloon on the clear box). Thus, the voltage curve drops are wider and deeper for fuller balloons. This experiment confirms that balloon volume gives rise to distinct patterns, which are conceivably detectable if multiple source-detector pairs are deployed at the right locations, and they are active at the same time

Note that volume estimation based on threshold values, even for this simple bladder phantom, may not always yield accurate results due to noise. The physical system of the disclosed technology, a human body and the bladder specifically, can have far higher variability across patients and is guaranteed to yield noisier measurements

Second Sample

Second bladder phantom includes two parts: a water-filled container representing the bladder; and a larger container filled with ground beef, representing the tissue surrounding the bladder. The water-filled container is placed inside of the surrounding tissue container to approximate the anatomical placement within the body. To mimic the bladder's natural expansion with increasing volumes of urine, two different sizes of water-filled containers are used.

The goal to address two missing pieces of information in the first sample. Specifically, 1) qualitatively demonstrate feasibility in presence of tissue, and 2) obtain quantitative results on spatial resolution for the system. To do this, the optical probe is fixed in place. Subsequently, the phantom is placed on the optical probe at a controlled location, and measurements of diffuse light are taken and averaged every 0.5 cm. The process is repeated many times, with different positional placement of the container.

The experiments are performed with two bladder-container sizes, represented by square water-filled containers of sizes 4×4×4 cm and 7×7×4 cm. The emitter is driven at 150 mA resulting in about 106.5 mW of optical radiation, and a fixed source-detector separation of 2.75 cm is used, which results in an effective depth of penetration of around 1.3 cm. The large absorption of 970 nm light in water result in a noticeable difference between the bladder location and surrounding tissue.

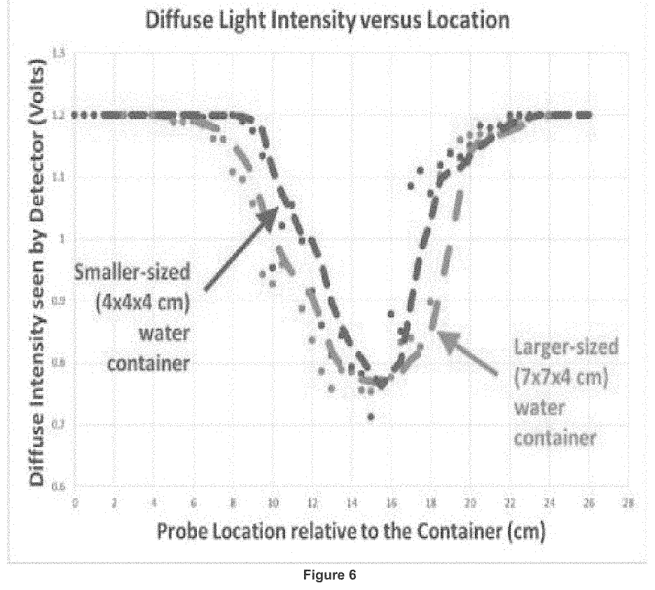

FIG. 6 illustrates incident diffuse light, as a voltage measurement, with respect to the relative probe-container position. As seen, the larger-sized bladder dropped off in about 100 mV equivalent light intensity around 1.5 cm before and after the smaller-sized bladder. Given the 3 cm difference between the two bladder sizes, the observed results are logical and expected. As the water-filled container begins to move above the source-detector, more light is absorbed by the water than in the tissue. There's a maximal absorption point at the center of the water container. The absorption tapers off as the container moves away from the optical components. These preliminary results support the hypothesis that changes in bladder volume can be detected using NIR-spectroscopy.

C. Sample for Human Subjects

The disclosed technology relates to a practical, non-invasive, and easy-to-use solution for estimation of bladder volumes with sufficient accuracy. An estimation accuracy is about 25 ml which is sufficient for the target application. Near-infrared spectroscopy may be used by the disclosed technology.

The disclosed technology relates to a flexible probe, including several inexpensive light-emitting diodes and photodetectors, which can be worn discreetly and comfortably on the lower-abdominal area under clothing, like an adhesive patch. The probe may be flexible for the most part, and to have an area of about twice that of a credit card, and be only a few millimeters thick. Attachment of optical probes using medical-grade self-adhesive patches is in clinical use in some applications, such as cerebral oximetry.

The disclosed technology relates to the disclosed system, including flexible optical probe with multiple light sources and photodetectors, analog interfacing circuitry and a battery-powered embedded computer, to activate light sources in sequence, and to collect correlated measurements from all detectors. The disclosed technology relates to a number of bladder phantoms using pig bladders with appropriate tissue layers between the probe and the bladder. The disclosed technology relates to a data-dependent machine learning algorithms, which is feasible by, e.g., quantifying the correlation between measured data and porcine bladder volumes.

There is an inherent tradeoff between the complexity and size of the optical probe (which also impacts the complexity of the interfacing circuitry) and the system's estimation accuracy. Particularly, the probe design strikes a balance between volume estimation accuracy vs. practicality from user's viewpoint (cost, size, etc.). Since there is no need to solve a complicated inverse problem using the measurements, the probe design may be significantly simplified compared to what is typically used in imaging techniques, such as diffuse optical tomography, which have to obtain solutions to complicated inverse problems.

To improve system efficacy for human subjects, the disclosed technology can make necessary adjustments to probe design and other aspects of the system that render the system a better fit for human subjects. Humans have a different body shape and size, and it is conceivable that optimizing probe design for humans, compared to phantoms, can require additional effort. This includes not only the number and size of optical elements, but also their specifications and those of the interfacing circuitry such as driving current, detector responsiveness, amplifier noise figures, etc. Furthermore, data are collected to enable construction and training of appropriate volume estimation algorithms.

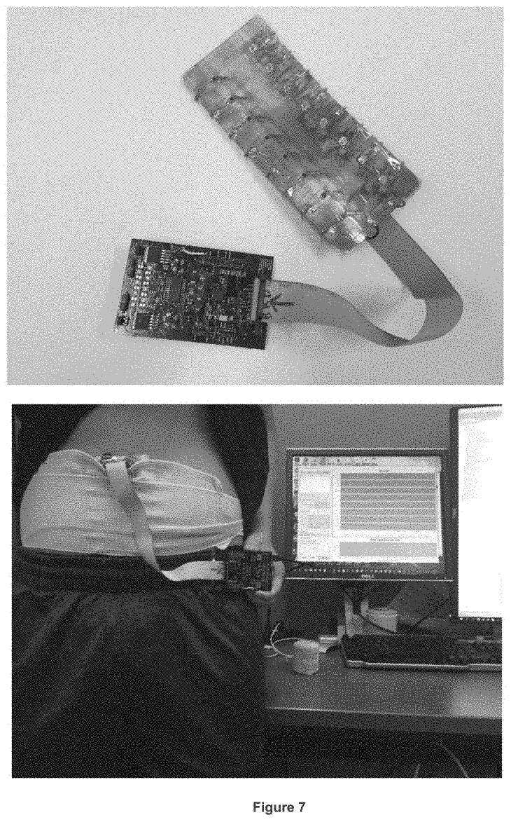

FIG. 7 illustrates a wearable sensing device and its application on a human subject, where a computer is interfaced with the wearable sensing device to capture and virtualize the data. The wearable sending device includes multiple optical elements mounted on a copper-coated polyimide flexible substrate. The flexible substrate allows the probe to bend easily, and to follow the contour of the subject's lower abdomen after attachment. The probe may be about 4 mm think, and is covered with a clear disposable medical-grade tape before attachment to body to allow hygienic and safe reuse between different subjects.

The illustrated device has 8 light emitting diodes (LEDs) and 8 photodetectors that are arranged in linear fashion. That is, all LEDs are placed in a line with 2 cm spacing between them, and similarly photodetector are placed on an opposing line with 2 cm inter-detector spacing. The spacing between corresponding LED and detector is 4 cm in this illustrated device.

The spacing between optical elements relates to device's sensitivity with respect to photons that have traveled to the appropriate depth of the body, and its ability to differentiate between an empty and full bladder. In some alternative embodiments, the spacing between optical elements are not fixed. For example, the spacing between optical elements can be different from each other by taking consideration of the physical contour of the lower abdomen area. The optical elements can be even separated on one or more flexible supports (e.g., copper-coated polyimide flexible substrates).

In some embodiments, the illustrated device can include any arbitrary number of light emitters or photodetectors. The number of the light emitters can be different from, or the same as, the number of the photodetectors. In other words, each of the light emitters can correspond to one or more photodetectors; each of photodetectors can also correspond to one or more light emitters.

In some embodiments, the light emitters and/or photodetectors can be arranged either in a linear fashion or nonlinearly (e.g., a 2-D pattern). For example, the photodetectors can be arranged in a 2-D pattern that covers a major portion of the surface area of the lower abdomen. Similarly, the light emitters can be arranged in a 2-D pattern that covers a major portion of the surface area of the lower abdomen.

The illustrated device connects to an interfacing circuitry, as illustrated in FIG. 7. The interfacing circuit connects to the probe on one end, and connects to a computer via USB connection on another end. It allows activation of the LEDs in sequence, reading out of sensor values at the right time, and their transfer to a software running on the computer for storage, analysis and visualization.

The circuit drives its power from the computer via the USB cable, and uses the USB power source to turn on the LEDs at a programmable current between 50 and 800 mA. The higher driving currents, which are confirmed to be safe (refer to Section IV), are provisioned for patients with substantial abdominal fat, as the optical signal needs to travel deeper in the tissues to reach the bladder.

In a test of the disclosed device, a healthy volunteer subject wears the device. The end of the probe is placed on the subject's pubic bone, and the other end of the probe ended up on the subject's belly button (FIG. 7). The device is turned on for about a minute, and data is collected at a frequency of 1 Hz (1 sample per second) before and after voiding his bladder.

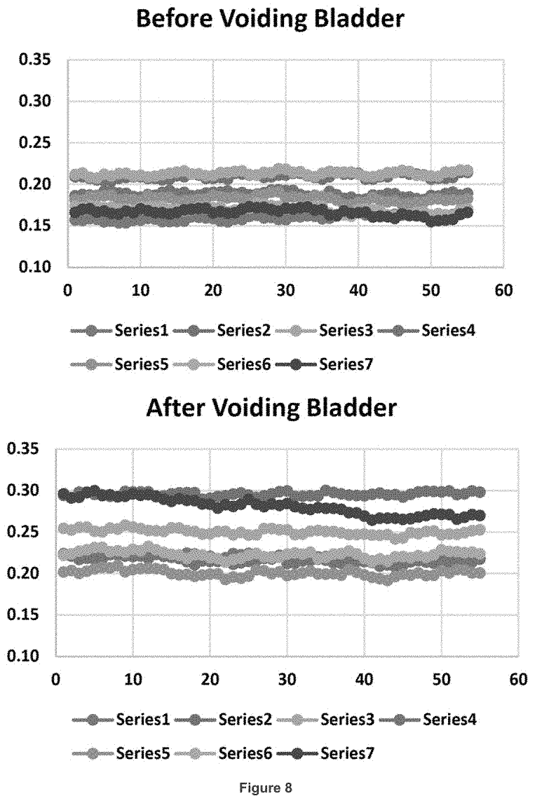

FIG. 8 illustrates diffuse intensity (volts) sensed at multiple photodetectors when the corresponding light emitting diode is turned on, before and after voiding the bladder. The numbers range 150-220 mv for full bladder, and 200-300 mv for empty bladder in the illustrated embodiment. While individual single-point measurements are noisy, there is a clear pattern of decreased light absorption (higher diffuse intensity) after voiding.

FIG. 8 illustrates the data obtained in this experiment. The top side refers to before voiding the bladder (full bladder), and the bottom side of the figure shows the data after voiding (empty bladder). Each plot shows seven voltage series data (corresponding to the intensity of diffused light—the lower the voltage, the lower the amount of diffused light sensed on the skin (more light absorbed by subject's body)) that are recorded by the lower 7 photodetectors: detector 1 (series 1 in the figure) is placed on the pubic bone, detector 2 (series 2) is the one immediately above that (2 cm higher) and so forth.

In some embodiments, the 8th and last detector produced numbers that are out of range (outliers), and thus, its data are removed from the chart for clarity. In some embodiments, this occurred as the 8th LED and photodetector ended up on the two sides of the subject's belly button, which allowed significant light to travel to the sensor without having to travel through the subject's body.

There are observations about the raw sensed data for the illustrated device.

Individual measurements from any detector are noisy and unreliable as a basis for bladder fill assessment (as expected), due to natural movements, breathing and so on. In addition, part of the abdominal volume that is occupied by a full bladder, can be occupied by intestines after voiding. Depending on the intestine composition, consumed food, bowel movements, and other unpredictable factors, light absorption from each LED can be impacted differently. It is impractical to quantitatively infer bladder volume from a single sensor reading.

Despite noise and variability, each colored time series (output of a single detector) has a relatively stable average, which indicates that the measurements have a strong underlying signal mixed with a relatively smaller additive noise that varies from one measurement to the next. The strong average signal (relatively small variance in each time series) supports repeatability of experiments.

All detectors sense a higher amount of diffuse light after bladder voiding. For example, series 1, 2 and 5 have an average of ˜150 mv, ˜200 mv and ˜180 mv before voiding, and ˜220 mv, 300 mv and ˜200 mv after voiding, respectively. This confirms the initial hypothesis that urine would absorb 970 nm light more than the tissue or organs that replace it after voiding.

There is a clear trend/pattern that exists in the data, which can be used as leverage to infer bladder state and its fill volume. While data from any single detector may be too noisy for reliable bladder fill assessment, the entire sensor arrays paints a clear picture of the bladder fill state.

Robust Volume Estimation from Noisy Measurements

Using the disclosed device, extensive trials can be performed on healthy subjects to collect data for building and training effective machine learning models for bladder volume prediction.

1. Feature and Algorithm

For each test, a vector of features is collected, which includes the measurements of all photodetectors, the corresponding LED that is turned on to make the measurements, and the measured bladder volume for that test (label). In addition to these features, other important features that might affect the prediction results are the gender, weight, height, abdominal circumference, age, and time of day (this may correlate with the posture of the person, the amount of liquid that is typically consumed, etc.). The numbers of LEDs and photodetectors needed are design considerations as these numbers can affect the data vector dimension as well as the physical footprint, cost, use convenience and ultimately, success of the approach.

The feature selection is closely related with algorithm selection. On the one hand, algorithms that require only simple calculations are preferred, but these algorithms may need more features, e.g., more LEDs and photodetectors, to achieve the desired accuracy level. On the other hand, more sophisticated algorithms may require a smaller number of features (and hence can reduce the physical footprint of the device) but may require complicated computations, which demand more energy and drain battery faster.

The disclosed technology can use, e.g., classification algorithms, which can output discrete outcomes such as alert (bladder volume is high enough) or no alert (bladder volume is too low). Algorithms such as logistic regression and linear support vector machine (SVM) can be used. Alternatively, more sophisticated nonlinear SVMs using kernel trick with different choices of kernels can be used. For each algorithm, the accuracy, the number of LEDs and photodetectors used the energy required to execute the classifier can be estimated.

In addition to classification, regression methods may also predict the bladder volume from the collected features. This can provide more information to users. Again, algorithms that use less computation such as linear regression can be used. Alternatively more sophisticated methods such as generalized linear regression and nonlinear regression methods.

2: Energy-Aware On-Demand Observation Management

Adaptive on-demand observation methods can also be used. The main idea is that, instead of performing testing at regular time intervals, algorithms can automatically and adaptively determine the next time instance for measuring diffuse optical signal by the device. The key observation is that when the bladder volume is too small or right after CIC, it is not necessary to take measurements frequently, so battery life can be saved. On the other hand, when the bladder volume is relatively high (but has not yet reached the alarm threshold yet) or if it is right after mealtime, it is prudent to take more frequent measurements so that the delay between the time the bladder volume has become large enough and its detection is minimized. Adaptive algorithms can use, e.g., the quickest detection with energy constraints framework.

In quickest detection with energy constraint framework, it is interested in detecting the occurrence of phenomena (in this case, detecting if the bladder volume is larger than a patient-specific threshold) based on noisy observations, with minimum delay under energy constraint. Noisy observations can be the diffuse optical signals measured by the photodetectors.

In the ideal case, to minimize the delay between the time when the bladder volume reaches the threshold and when an alarm is generated, the system can continuously take samples. However, the energy cost of continuously taking observations and associated calculations render this an impractical solution.

The detection problem can be solved under energy constraint. The energy constraint presents several challenges and unique features to quickest detection problems. On the one hand, it is not desirable to take two successive observations too closely as this can cost energy before enough change occurs in the physical system (bladder). On the other hand, it is not desirable to make the time interval between two successive measurements too large as this strategy may introduce excessive detection delays. Furthermore, as the system cannot take samples most of the time, it may have missed data. Adaptive sensing strategies rely on information extracted from samples taken in the past, to make upcoming sample and detection decisions. Towards this end, using tools from optimal stopping theory, the system solves the following optimization problem:

$${min}_{T , μ} [ Pr ( T < λ ) + cE \{ {( T - λ )}^{+} \} ]$$

$$s . t . {\sum }_{t = 1}^{k} {μ}_{t} \leq N , ∀ k .$$

In this optimization problem λ is the time when the bladder volume crosses the threshold, T is the time when an alarm is raised, T−λ is the detection delay, which the system can like to minimize, and μ denotes sampling policy. The system aims to an optimal sampling policy μ and alarm strategy T to minimize a tradeoff between the probability of false alarm Pr (T<λ) and detection delay T−λ, subject to energy constraints.

In some embodiments, if the distribution of signal is not dynamic, even with limited number of samples, the scheme can achieve performance that is comparable to the ideal scenario in which, one can take samples continuously. The performance of the scheme can be significantly better than periodic sampling, and is very close to the continuous sampling policy (which is too energy-hungry to perform).

In bladder volume estimation, the distribution of signal is dynamic. The above optimization problem is solved and the corresponding performance is analyzed, under bladder-relevant dynamic signal distribution assumptions.

3: Personalization and Incremental Customization

Given the large variation in patient's body form and other relevant factors, it is conceivable that a one-size-fits-all policy may fail to smoothly work for all patients. To address this issue, the technology uses an algorithm to incorporate user's feedback to continuously improve device's prediction accuracy for its user. Once the device is in use, the user can optionally give feedback on (a subset of) alarms, e.g., via a smart watch, to indicate whether a specific prediction is useful. This may be a 1-bit “usefulness” feedback and there is no need to rely on urine volume feedback, however, target patient population can be routinely asked to keep CIC urine volumes diaries (to evaluate regimen changes, such as a new medication). The disclosed technology can take advantage of this valuable data, whenever available.

The disclosed device can automatically record other features, such as sensor measurements and time of the day. After each user feedback, one additional vector of labeled training data can be obtained. The idea of incremental learning can be used to improve the predictive model. In particular, the disclosed technology can use incremental SVM to adjust the SVM model. For other learning algorithms mentioned above, such as logistic regression, stochastic gradient descent algorithm can be used to fine tune the model with the sample. Thus, the performance of the disclosed device can be improved gradually during the use of the disclosed device.

Depending on the available computing and battery resources, the disclosed technology can balance the tradeoff of executing incremental learning 1) locally on the device at the time of feedback, 2) on the device but delayed to when sufficient energy resource is available (connected to charger), 3) remotely on a server per feedback, 4) batch communication with the server after several feedback vectors are available. For example, the disclosed technology can use low complexity schemes related to stochastic gradient descent algorithms to reduce the implementation complexity.

Evaluation

A. Lab Prototypes

As a first evaluation, the simple bladder replica prototype can be enhanced by wrapping the balloon in tissue-like material that can be easily purchased from a grocery store, e.g., a piece of steak with layers of muscle and fatty layers. The bladder is situated very close to the skin (2-4 cm deep) in humans. The tissue-replicas need to have the same thickness for fair comparison.

Next, porcine bladders can be obtained (e.g., from UC Davis meat lab) to use in lab prototypes, and to evaluate the system in a more realistic bladder replica. The system can be evaluated on 15 different porcine bladders to observe the impact of variability in factors such as bladder size, bladder wall thickness, and tissue layers, on the performance of the system. The purpose is to improve all components of the sensing system to demonstrate efficacy of the approach for lab prototypes, before proceeding to involve human subjects in the project.

B. Evaluation in Human Subjects

The system can be further evaluated on human subjects. The device can emit low amount of near infrared light at low energy values, which is safe in health applications, and is widely used in commodity technologies such as pulse oximetry. The technology can drive light emitting diodes in sequence, and at 50-150 mA. Measurements are few and far in between: tens of millisecond on and minutes off. Therefore, the amount of emitted energy and generated heat in the subject's body can be far lower than safe limits.

For example, sequentially work can be performed with two groups of subjects. First, 50 healthy volunteers are recruited to participate in evaluation of the system. After each data collection with the device, healthy subjects are asked to volitionally void their bladders into a measuring cup. Catheter is not used to empty their bladder. For healthy subjects who feel that they cannot completely void their bladders (those who sense urine is left in the bladder after voiding), the test can be carried out in the urology clinic, and can perform ultrasound imaging of the bladder to objectively measure the bladder volume after voiding. Ultrasound imagers are regularly used at bedside in the clinic.

Given the sensitivity of optical measurements to distance, for each patient, the probe is placed at several different nearby positions in the lower abdominal area, which records all such measurements. The rationale is to replicate reasonable use cases of the system, as during eventual deployment, the probe may be placed not at the same exact position every time, or it may slightly move due to patient movements and such. Every subject can be invited to participate repeatedly, at different bladder fill volumes.

The system is also evaluated in 20 SCI patients who are already scheduled to undergo urodynamic testing. Evaluation during the urodynamic test can allow the technology to collect data at various bladder volumes as water is being instilled into the bladder.

Sample Device Components

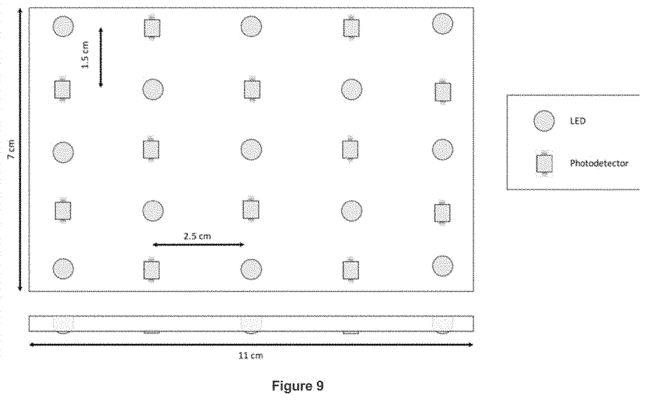

According to at least some embodiments of the disclosed technology, FIG. 9 illustrates a sensing system that includes a non-invasive optical probe patch, electronic circuitry, and a software module for analyzing collected data.

The non-invasive optical probe patch (also referred to as optode patch) serves as the patient interface for the sensing system. The patch may include material such as a flexible silicone rubber substrate. The substrate is embedded with one or more embedded light-emitting diodes (LEDs) and one or more photodetectors on a copper-coated polyimide sheet for monitoring the bladder using light-based measurements. At least portions of the substrate that come in contact with the patient are covered with polyimide to prevent skin-irritation. The optical probe patch can be, e.g., approximately 7 cm×11 cm×0.5 cm in size and can be attached to the patient's lower abdominal area using medical-grade adhesives. Both the LEDs and photodetectors can be encapsulated in silicone resin and are safe to skin contact.

On/off states of the photodetectors and LEDs can be controlled by an electronic unit, which is connected to the optode patch by, e.g., wires. Thus, the electronic unit does not need to contact the patient. The electronic unit can be fully encapsulated within an enclosure to protect the electronics and prevent any accidental damage to the device or harm to the patient. The power source of the sensing system can be attained from, e.g., a USB connection to a computer (which powers at 5V via the USB hardware protocol). Photodetectors are passive devices that measure the amount of light that is detected and generates very low current (e.g., on the order of several microamps). The LEDs emit light with, e.g., a 970 nm wavelength, a low-energy light wave. To monitor the bladder, a measurement sample can be taken at a rate of 1 Hz (1 sample per second). Each measurement is performed by turning the LEDs on sequentially in a “Round Robin” fashion with a maximum of 900 mA current for very brief periods of time (total time to cycle through all LEDs being less than 100 ms). One LED can be on at any time during the operation. In other words, no two or more LEDs are turned on at the same time. Measurements can be taken every second (sampling rate of 1 Hz) and the LEDs are on for about 100 ms of that time (duty cycle of 10%). For example, if the optode patch includes 20 LEDs, each LED is on for about 5 ms during each cycle.

The safety of the sensing system can be confirmed by comparing the maximum possible light emission output with the international standard IEC 60601-2-57/2012. The sensing system can satisfy specifications for the basic safety and essential performance of non-laser light source equipment intended for therapeutic, diagnostic, monitoring, and cosmetic/aesthetic use.

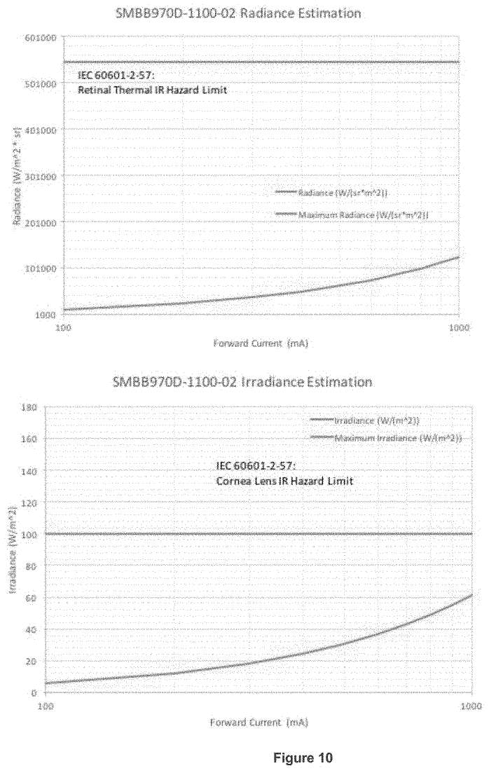

For achieving the maximum levels of light emission possible, the sensing device can be within the specifications for the exempt group classification for the Cornea Lens IR Hazard and Retinal Thermal IR Hazard limit (the two hazards that is applicable for the optode patch—note that these specifications are conservatively defined for radiation into the eye for the worst possible scenario). FIG. 10 illustrates a comparison between radiance and irradiance limits and the disclosed sensing system. The above lines represent the thresholds for being classified in the exempt group under IEC 60601-2-57 for the Retinal Thermal IR Hazard limit and the Cornea Lens IR Hazard limit. The upper limit values of the radiation and irradiance of the disclosed sensing system (as if one LED is constantly on) is well below these limits. For reference, the probe LEDs are driven with current values less than 900 mA and a total LED on duty cycle of at most 10% (maximum of 100 ms of activity in every second). Note that the maximum time of light emission from the LEDs per sample measurement is 100 ms, so the amount of energy received by the patient is at most 10% of the lower curve shown in FIG. 10, which clearly shows that the energy emitted from the probe cannot harm the patient.

The electronic circuitry can include a printed-circuit board, which hosts various data acquisition electronics such as microcontrollers and analog-to-digital converters. The microcontroller controls the optical probe patch to acquire data periodically (˜1 measurement per second). The electronic circuitry can be approximately 5 cm×10 cm in size. The electronic circuitry can be fully enclosed in a container (e.g., a 3D-printed container) to protect it from the environment and patient. The electronics and optode patch can be powered via a USB-cable from a computer or a power supply which carries a maximum voltage of 5V. For example, the USB 3.0 specifications identify that high-power devices when operating in SuperSpeed mode can draw a maximum of 900 mA. If the current rises above this limit, current-limiting mechanisms in conventional computers turn the USB port off, and thus all power to the bladder sensor system, which can only be re-enabled by a hard-reset. This acts as an ultimate fail-safe in the hypothetical scenario that the device malfunctions and the LEDs try to draw more than 900 mA. It is noted that this hard 900 mA current limitation caps the maximum light emission possible by the disclosed system. Furthermore, the circuitry does not come in to contact with the patient and poses little to no risk. The data collected by the data acquisition circuitry will be sent to a software running on a computer via USB to save for post-processing. No personal identification data will be retained in these measurements. The usage of the device for the disclosed system can be performed in the urology clinic with medical staff at hand for approximately 30 minutes.

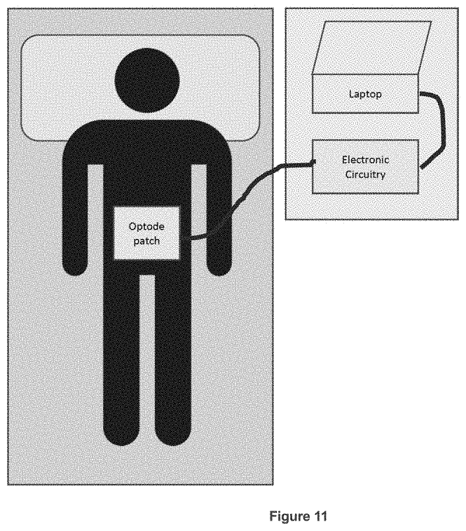

FIG. 11 illustrates a sample scenario that the sensing system is used to monitor a patient's bladder. In some embodiments of the disclosed technology, the usage of the disclosed system involves: 1. placing the optode patch on the skin of the patient in the lower abdominal area near the bladder. 2. connecting the patch to the electronic circuitry via fully insulated wires. The electronic circuitry can be placed near the patient, e.g., on the bedside, table. 3. connecting the electronic circuitry to the computer running the software module via a USB cable (may also be placed near the patient). 4. using the software module running on the laptop to initiate data acquisition.

Sample Device Architecture

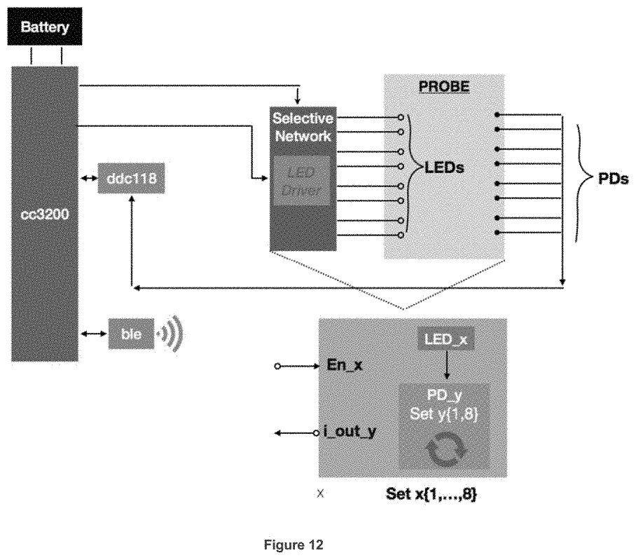

In some embodiments of the disclosed technology, a bladder volume sending system includes a CC3200 Texas Instrument (TI) Launchpad micro-controller to execute the control logic of the bio-medical device, a selective network to control 1 of 8 LEDs, an LED Driving component to deliver high electrical current to power the LEDs, a data acquisition component to capture the light signal intensity detected by eight photodiodes, and a Bluetooth Low Energy (BLE) component to transmit wireless data. In some embodiments of the disclosed technology, the battery capacity can sustain the device operating for a 12-hour battery life span. FIG. 12 illustrates a sample architecture of a bladder volume sensing system.

In some embodiments, the interface for data communication can be either wireless or wired. For example, the data communication interface can be a Bluetooth or Wi-Fi interface, or a wired Ethernet interface. In some embodiments, various components of the system (e.g., process, light emitters, sensors, data communication interface) can be implemented as an embedded device. The embedded device can be embedded inside of the body of the patient.

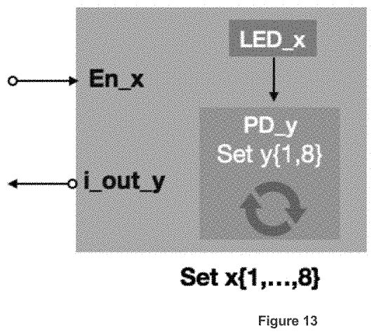

The TI CC3200 launchpad board can be the master Micro-Controller Unit (MCU) that executes the logic for the entire embedded system. The goal of the MCU is to emit light photons from an LED aimed into the bladder region of a human subject and detect, through a set of photodiodes, how much of the light exits the region relative to the initial emitted light. FIG. 13 illustrates a high-level overview of the control logic. Let x and y serve as a specific LED and photodiode on the probe block in FIG. 12 respectively. The control logic enables LED x using the Enable input port (EN_x) to start emitting light photons. While the LED is turned on, a measurement reading from eight photodiodes is taken and shifted out in series through the output port (i_out_y). If the light signal detected by any photodiode is saturated then the LED operating current is adjusted and the process is repeated until a desirable signal is obtained for LED x. This logic is repeated for every LED located on the probe. Data is transmitted through the wireless BLE communication channel as it becomes available.

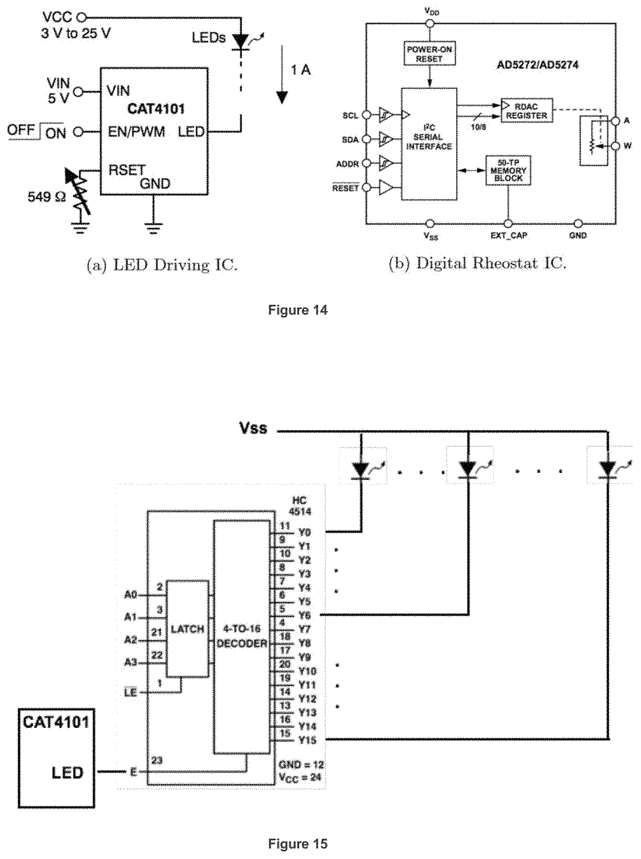

FIG. 14 illustrates an LED Driver IC and a Digital Rheostat IC that can be used together to achieve dynamic LED sink current levels. The probe LEDs can be operating at low to high electrical current levels in order to emit a large quantity of light photons into the bladder region. The CAT4101 is an LED Driving IC that provides a constant-current sink up to 1 A with a low dropout of 0.5 V at full load. The VIN and GND pins on the LED Driver are used to power the IC while the EN/PWM pin is used to enable the LED driving pin, and the RSET pin is used to adjust the current-sink level by varying the resistance between RSET and GND.

Current-sink adjustment can be done programmatically by connecting a Digital Rheostat IC (AD5272) between RSET and GND on the LED Driver. With the help of these two ICs, the MCU can dynamically adjust the operating LED electrical current until a desirable signal is obtained from the photodiodes. The Digital Rheostat (AD5272) communicates with the MCU over an i2c communication channel using the SCL, SDA, and ADDR pins.

FIG. 15 illustrates an LED selective network. The unique output (Y0-Y15) can be selected to operate an LED with the current-sink level set in the CAT4101 IC. Multiple LEDs are located throughout the bio-medical probe, but the MCU is used to turn ON one LED while the remaining LEDs are turned OFF, so the light signal detected by the photodiodes is from a single LED source. To achieve this desired mechanism a high-speed CMOS Logic 4-to-16 Line Decoder/Demultiplexer with Input Latches IC (CD74HC4514) can be controlled by the MCU to select one from many LEDs.

Notice the current-sink LED pin from the LED Driver component is fed into a pin of the Demultiplexer IC as shown in FIG. 15. This configuration allows the selected output LED to be driven at the current-sink level set in the LED Driver. The four Demultiplexer inputs (A0-A3) are used by the MCU as a binary truth table sequence to select and enable 1 of 16 outputs (Y0-Y15). Each output is connected to a unique LED. In this system architecture, 8 outputs are utilized since it's the total LED count of the probe design, according to some embodiments.

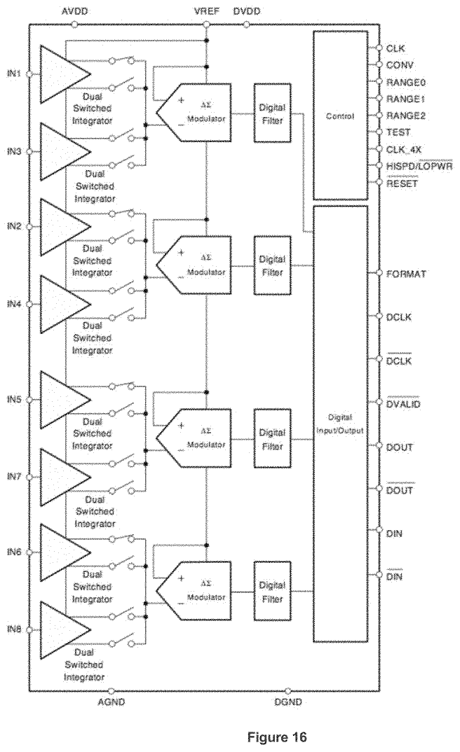

FIG. 16 illustrates a sample IC (DDC118) that controls the photodiodes. The DDC118 IC allows the MCU to collect light signals from eight different photodiodes at the same time. When an LED is turned ON, the MCU needs to read the amount of light signal detected by eight differently spaced photodiodes. This is done using the DDC118 IC which takes in eight photodiodes as inputs (IN1-IN8) and serially shifts out the ADC converted light signal value detected by each photodiode through the DOUT pin. All other pins on this IC are used to configure and power the IC. The DDC118 IC integrates all inputs for a specific duration of time to collect some charge, from each input, and determine the amount electrical current released by each photodiode. A conversion of each photodiode current can be performed to calculate the light intensity each photodiode detected since they are directly proportional, thus arriving at the MCU's goal.

In short, pins RANGE0-RANGE2 of the DDC118 are used to programmatically select a capacitor size. The integrators use this capacitor to collect the photodiode input charge for a given integration time specified by the CONV pin. Holding the CONV pin high can trigger the integrators to operate. All eight photodiode measurements can be shifted out in series through the DOUT pin once they have been integrated and converted by their ADCs (Modulator). The DDC118 can internally pull the DVALID pin low, advising the MCU that data is ready, as soon as all of the integrators and ADCs have completed their operation.

A valid integration time can be set from a range of 50 μs to 1000000 μs and the available capacitor charge size ranges in increments of 50 pC from zero (12 pC) to 350 pC. With these specifications the largest DC electrical current value that the DDC118 can measure is 350 pC/50 μs=7 μA, thus values above 7 μA can saturate the ADCs. This system architecture can be configured with a 350 pC capacitor and integration time of 50 μs allowing data to ready every 400 μs (50 μs×8 photodiodes).

Phantom and Ex Vivo Experiments and Results

According to some embodiments, a NIRS measurement system is disclosed to capture the diffuse reflected light that can investigate the underlying tissue composition. The optical components include one or more high-power LEDs and one or more monolithic silicon photodiodes, placed 4 cm apart from each other. The photodiode can be connected to a transimpedance amplifier and low-pass filter before being digitized using a 22-bit analog-to-digital converter. The electronics interact with custom-written software for data capture, analysis, and real-time visualization of the detected light intensity.

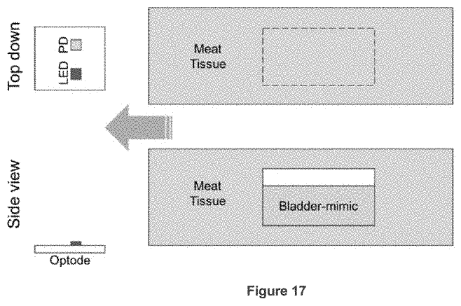

FIG. 17 schematically illustrates an experimental setup including an optical phantom. The optical phantom is slid laterally over the optode in 1 cm increments. The optical phantom is to mimic the gross anatomy and optical properties of the bladder and its surrounding environment. As shown in FIG. 17, the bladder is represented with an 8 cm container filled with water, and its surrounding tissue is represented using a mixture of bovine muscle and fat with a thickness of 2 cm between the optical probe and the bladder representation.

The optical phantom is used with the NIRS measurement system to collect the light intensity profile over the length of the tissue model. This is accomplished by moving the phantom laterally across the optode in 1 cm increments, where at each interval, the LED is driven with a constant current and the diffuse-reflected light seen at the photodiode is measured and analyzed.

The system is tuned with the wavelength to attain an appropriate level of sensitivity for measuring a reasonable optical signal. In some embodiments, three LEDs with peak wavelengths at 890 nm, 970 nm, and 1450 nm can be used respectively. These wavelengths are chosen to investigate the effect that different absorption coefficients have on the overall light intensity measured, in addition to the coefficient's stability over small variations in wavelength, which helps to reduce errors caused by slight shifts of the peak wavelengths in the LEDs. The absorption coefficient for water at 890 nm is 0.058 cm⁻¹, 970 nm is 0.481 cm⁻¹, and 1450 nm is 32.778 cm⁻¹. All three LEDs are driven at 200 mA.

As the bladder fills with urine, the optical signal tends to attenuate. To test the attenuation, the aforementioned protocol is performed for three different volumes of liquid, namely 100 ml, 300 ml, and 500 ml, since the normal human bladder has a capacity around 400-500 ml. An LED with a peak wavelength at 970 nm (200 mA) is used.

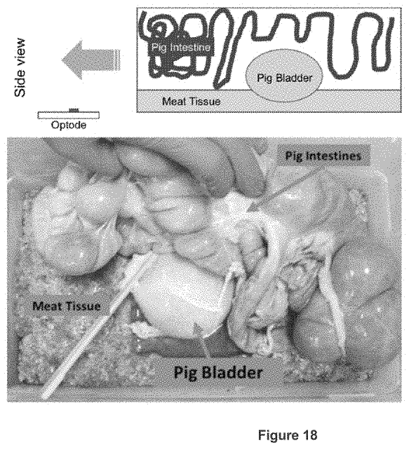

As a step towards investigating a more realistic bladder environment, a pig bladder and intestines immediately post-mortem are used to ex vivo measurements using the disclosed system. FIG. 18 illustrates an ex vivo experimental setup using a pig bladder and intestines to create a more realistic tissue model. As illustrated in FIG. 18, the bladder is surrounded by the intestines in order to create a more anatomically accurate depiction of the bladder environment. Using a system of syringes, tubes, and clamps, the pig bladder is filled with 200 ml of water and the 970 nm LED (670 mA) is used to perform the measurements.

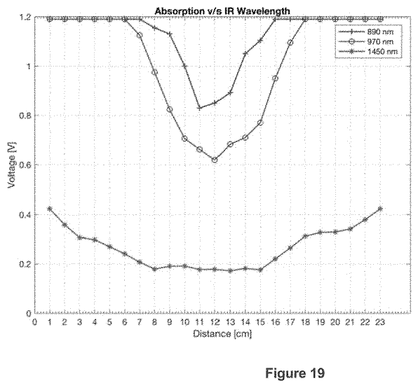

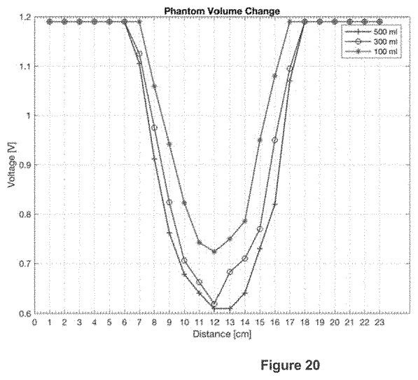

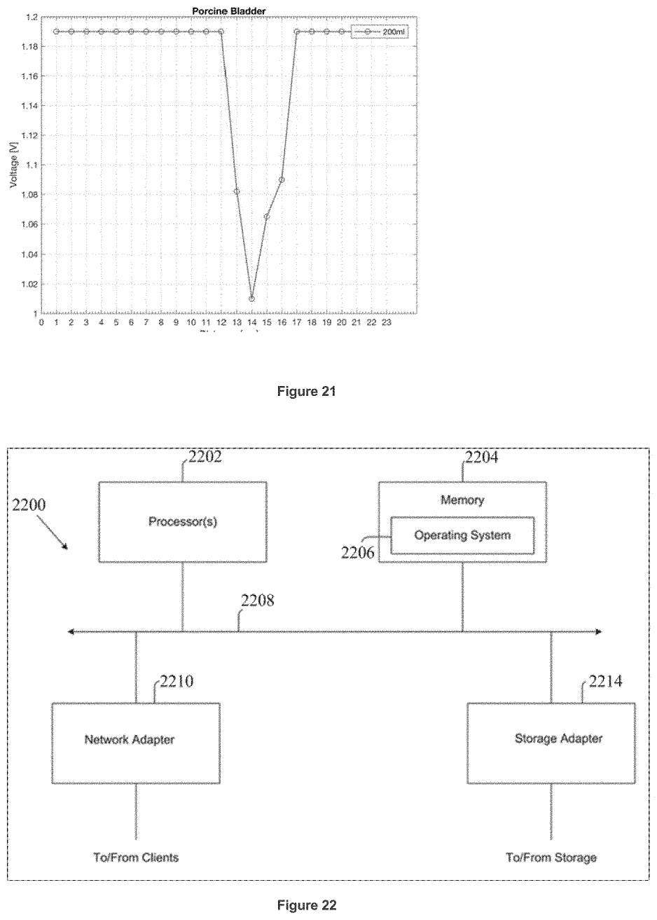

FIG. 19 illustrates measurements performed on the optical phantom over three wavelengths (890 nm, 970 nm, 1450 nm). The depth of the light intensity signal (in Volts) follows the absorption coefficient for water at these wavelengths. FIG. 20 illustrates measurements performed on the optical phantom over three volumes of liquid (100 ml, 300 ml, 500 ml) using a 970 nm LED. As the amount of water in the bladder increases, the light intensity drops. FIG. 21 illustrates the ex vivo measurements performed on a pig bladder (filled to 200 ml of water) using a 970 nm LED. As the NIR light field passes over the bladder, the characteristic drop in light intensity appears.

Measurements taken over different wavelengths can be seen in FIG. 19. The y-axis shows the voltage measured by the ADC, which represents the light intensity seen at the photodiode. The x-axis shows the spatial location of where the measurements are taken in relation to the optical phantom. The bladder, represented by a container of water in this case, is located between the 8 cm and 16 cm marks on the graph. As the NIR light passes over the bladder, there is a drop in the light intensity for all wavelengths. However, the change in light intensity for 890 nm light shows the least amount of attenuation to the presence of the bladder, whereas 970 nm light shows a more significant change. Since 1450 nm light has such a strong absorption coefficient, it can be difficult to assess when the probe passed over the bladder. In other words, the 1450 nm light signal can be too sensitive. The 970 nm wavelength provides an appropriate signal sensitivity and is selected for use in subsequent experiments.

As the volume of urine increases in the bladder, the light intensity can decrease due to the rising concentration of water in the tissue that light investigates. These measurements can be seen for different volumes of liquid in FIG. 20. Similarly, the bladder is located between 8 cm and 16 cm. As the volume of liquid in the bladder container increases, the light intensity measured by the detector decreases. This shows that there is a noticeable change in the light intensity signal that can allow us to distinguish between varying amounts of liquid in the bladder.

To represent a more realistic tissue model, an ex vivo measurements is performed using a pig bladder and intestines. When the NIR light field passes over the pig bladder, there is a noticeable drop in the light intensity seen by the photodiode, demonstrating the feasibility to obtain a reasonable signal using the disclosed setup with a 970 nm LED. This demonstrates the feasibility of using the system on humans.

FIG. 22 is a high-level block diagram illustrating an example of a hardware architecture of a computing device 2200 that may perform various processes as disclosed, according to various embodiments of the present disclosure. The computing device 2200 may execute some or all of the processor executable process steps described herein. In various embodiments, the computing device 2200 includes a processor subsystem that includes one or more processors 2202. Processor 2202 may be or may include, one or more programmable general-purpose or special-purpose microprocessors, digital signal processors (DSPs), programmable controllers, application specific integrated circuits (ASICs), programmable logic devices (PLDs), or the like, or a combination of such hardware based devices.

The computing device 2200 can further include a memory 2204, a network adapter 2210, and a storage adapter 2214, all interconnected by an interconnect 2208. Interconnect 2208 may include, for example, a system bus, a Peripheral Component Interconnect (PCI) bus, a HyperTransport or industry standard architecture (ISA) bus, a small computer system interface (SCSI) bus, a universal serial bus (USB), or an Institute of Electrical and Electronics Engineers (IEEE) standard 1394 bus (sometimes referred to as “Firewire”) or any other data communication system.

The computing device 2200 can be embodied as a single- or multi-processor storage system executing an operating system 2206 that can implement various modules as disclosed. The computing device 2200 can further include graphical processing unit(s) for graphical processing tasks or processing non-graphical tasks in parallel.

The memory 2204 can comprise storage locations that are addressable by the processor(s) 2202 and adapters 2210 and 2214 for storing processor executable code and data structures. The processor 2202 and adapters 2210 and 2214 may, in turn, comprise processing elements and/or logic circuitry configured to execute the software code. The operating system 2206, portions of which is typically resident in memory and executed by the processors(s) 2202, functionally organizes the computing device 2200 by (among other things) configuring the processor(s) 2202 to invoke. It will be apparent to those skilled in the art that other processing and memory implementations, including various computer readable storage media, may be used for storing and executing program instructions pertaining to the disclosed technology.

The network adapter 2210 can include multiple ports to couple the computing device 2200 to one or more clients over point-to-point links, wide area networks, virtual private networks implemented over a public network (e.g., the Internet) or a shared local area network. The network adapter 2210 thus can include the mechanical, electrical and signaling circuitry included to connect the computing device 2200 to the network. Illustratively, the network can be embodied as an Ethernet network or a Fibre Channel (FC) network. A client can communicate with the computing device over the network by exchanging discrete frames or packets of data according to pre-defined protocols, e.g., TCP/IP.

The storage adapter 2214 can cooperate with the storage operating system 2206 to access information requested by a client. The information may be stored on any type of attached array of writable storage media, e.g., magnetic disk or tape, optical disk (e.g., CD-ROM or DVD), flash memory, solid-state disk (SSD), electronic random access memory (RAM), micro-electro mechanical and/or any other similar media adapted to store information, including data and parity information. The storage adapter 2214 can include multiple ports having input/output (I/O) interface circuitry that couples to the disks over an I/O interconnect arrangement, e.g., a conventional high-performance, Fibre Channel (FC) link topology.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. The term “substantially coplanar” can refer to two surfaces within micrometers of lying along a same plane, such as within 40 μm, within 30 μm, within 20 μm, within 10 μm, or within 1 μm of lying along the same plane.

As used herein, the singular terms “a,” “an,” and “the” may include plural referents unless the context clearly dictates otherwise. In the description of some embodiments, a component provided “on” or “over” another component can encompass cases where the former component is directly on (e.g., in physical contact with) the latter component, as well as cases where one or more intervening components are located between the former component and the latter component.

While the present disclosure has been described and illustrated with reference to specific embodiments thereof, these descriptions and illustrations are not limiting. It should be understood by those skilled in the art that various changes may be made and equivalents may be substituted without departing from the true spirit and scope of the present disclosure as defined by the appended claims. The illustrations may not necessarily be drawn to scale. There may be distinctions between the artistic renditions in the present disclosure and the actual apparatus due to manufacturing processes and tolerances. There may be other embodiments of the present disclosure which are not specifically illustrated. The specification and the drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it can be understood that these operations may be combined, sub-divided, or re-ordered to form an equivalent method without departing from the teachings of the present disclosure. Accordingly, unless specifically indicated herein, the order and grouping of the operations are not limitations.

What is claimed is:

1. A system for sensing bladder volume, comprising:
at least one patch configured to attach to a human skin or a wearable garment at locations in proximity to an abdomen area;
a plurality of light emitters directed towards the abdomen area;
a plurality of light sensors configured to receive light signals that are emitted by the light emitters, reflected by human tissues, and transmitted through an abdominal wall, wherein at least one of the light emitters or at least one of the light sensors is disposed on the at least one patch; and
a processor configured to receive information of the received light signals and to predict a bladder volume based on the information of the received light signals.

2. The system of claim 1, wherein the processor predicts the bladder volume by using a machine learning model to identify patient-specific spatial and temporal patterns across light signals received by the light sensors.

3. The system of claim 2, wherein inputs of the machine learning model include the light signals received by the light sensors and additional attributes related to a patient or an environment.

4. The system of claim 3, wherein the additional attributes include patient weight, size of an abdominal area, fluid intake of the patient, temperature, or weather.

5. The system of claim 3, wherein the additional attributes include personal characteristic information.

6. The system of claim 2, wherein the machine learning model improves performance of the system by a process of incremental learning during use of the system.

7. The system of claim 1, wherein the plurality of light emitters are configured to activate in sequence.

8. The system of claim 1, wherein any of the plurality of light sensors is configured to received light signals that are emitted by any of the plurality of light emitters.

9. The system of claim 1, further comprising:
an alarming component configured to prompt to empty a bladder in response to the bladder volume exceeding a threshold value.

10. The system of claim 9, wherein the alarm component is configured to provide an auditory, visual, or vibratory prompt.

11. The system of claim 1, further comprising:
a wireless signal receiver implanted under the human skin and configured to receive the information of the received light signals and to relay the information of the received light signals to the processor.

12. The system of claim 1, wherein the processor is further configured to analyze the information of the received light signals by comparing the received light signals with light signals of catheterized or voided bladder volumes during a training period.

13. The system of claim 1, wherein the processor is further configured to instruct the light emitters or the light sensors to turn on or off based on user patterns for power saving.

14. The system of claim 1, wherein the processor is configured to predict the bladder volume further based on factors including one or more environmental factors or patient activity information.

15. The system of claim 1, wherein the processor is configured to predict the bladder volume using a machine learning model that has been trained using a training data set that includes light signal data that are flagged regarding bladder volumes.

16. The system of claim 1, wherein the light sensors are configured to emit infrared light having a wavelength of about 975 nanometers.

17. A wearable device for bladder volume sensing, comprising:
a flexible substrate;
a plurality of light emitters amounted on the flexible substrate; and
a plurality of light sensors mounted on the flexible substrate, wherein the sensors are configured to receive light signals that are emitted by the light emitters, reflected by human tissues, and transmitted through an abdominal wall.

18. The wearable device of claim 17, further comprising:
an electronic interface configured to transfer the light signals received by the light sensors to an external device for processing.

19. The wearable device of claim 17, further comprising:
a clear disposable tape covering the light emitters, wherein the clear disposable tape is configured to be detached from the light emitters before the wearable device attaching to an abdominal area of a human subject.

20. The wearable device of claim 17, wherein the light emitters are arranged in a linear fashion.

21. The wearable device of claim 17, wherein the light sensors are arranged in a linear fashion.

22. The wearable device of claim 17, wherein the flexible substrate includes a copper-coated polyimide sheet.

23. The wearable device of claim 17, wherein the flexible substrate includes a silicone rubber material.

24. The wearable device of claim 17, wherein the light emitters are configured to turn on sequentially.

25. A system for bladder volume sensing, comprising:
a wearable device, including:
a flexible substrate,
a plurality of light emitters amounted on the flexible substrate, and
a plurality of light sensors mounted on the flexible substrate, wherein the sensors are configured to receive light signals that are emitted by the light emitters, reflected by human tissues, and transmitted through an abdominal wall; and
a computing device electronically coupled to the light sensors of the wearable device, wherein the computing device is configured to receive information of the received light signals and to predict a bladder volume based on the information of the received light signals.

26. The system of claim 25, wherein the computing device predicts the bladder volume by using a machine learning model to identify patient-specific spatial and temporal patterns across light signals received by the light sensors.

27. The system of claim 26, wherein inputs of the machine learning model include the light signals received by the light sensors and additional attributes related to a patient or an environment.

28. The system of claim 27, wherein the additional attributes include patient weight, size of an abdominal area, fluid intake of the patient, patient activity, temperature, or weather.
